(19) United States
(12) Patent Application Publication
(10) Pub. No.: US 2016/0166479 A1
(43) Pub. Date: Jun. 16, 2016

(54) USE OF TRIALKOXYSILANE DERIVATIVES TO MODIFY THE APPEARANCE OF EMULSION-TYPE COSMETIC COMPOSITIONS

(71) Applicant: L'OREAL, Paris (FR)

(72) Inventors: Catherine CHIOU, Saddle Brook, NJ (US); Andrew B. GOLDBERG, Springfield, NJ (US); Angelike A. GALDI, Westfield, NJ (US)

(21) Appl. No.: 14/571,798

(22) Filed: Dec. 16, 2014

(51) Int. Cl. A61K 8/06; A61K 8/58; A61Q 19/00; A61K 8/37; A61K 8/49; A61K 8/891; A61K 8/893

(52) U.S. Cl. CPC A61K 8/064; A61K 8/891; A61K 8/58; A61K 8/893; A61K 8/37; A61K 8/4993; A61Q 19/00

(57) ABSTRACT

The present invention is directed to a translucent water-in-oil emulsion which comprises an aqueous phase comprising a least one neutralized trialkoxysilane derivative as an appearance modifier and an oil phase comprising at least one water-in-oil emulsifier.

### FIELD OF THE INVENTION

The present invention is directed to a water-in-oil type emulsion that is translucent in appearance. More specifically, the present invention is directed to a water-in-oil type emulsion comprising an aqueous phase comprising a least one neutralized trialkoxysilane derivative as an appearance modifier and an oil phase comprising at least one water-in-oil emulsifier.

### BACKGROUND OF THE INVENTION

The incorporation of trialkoxysilane derivatives into emulsion-type cosmetic compositions so as to alter their appearance from a conventional opaque look to a cleaner, more translucent look, poses various challenges. Compositions containing trialkoxysilane derivatives are unstable as demonstrated by changes in physical appearance and texture during storage at normal temperatures. When added to water, trialkoxysilane derivatives give rise to a high pH which is unsuitable for use in cosmetic products, and solutions of trialkoxysilanes in water must be neutralized to about pH 5 to about pH 6 for use in cosmetic products. Further, trialkoxysilane derivatives are incompatible with commonly used emulsifiers and thickeners, which adversely impacts stability of the cosmetic composition. These challenges result in a need to formulate stable cosmetic compositions comprising trialkoxysilane derivatives which are visually pleasing to consumers, without any negative impact on the sensorial texture of the composition.

### BRIEF SUMMARY OF THE INVENTION

In an exemplary embodiment, a composition in the form of a stable, translucent water-in-oil type emulsion containing at least one neutralized trialkoxysilane derivative and at least one water-in-oil emulsifier is provided. The composition provides a consumer-pleasing texture upon application. The composition includes an aqueous phase and an oil phase. The aqueous phase contains at least one neutralized trialkoxysilane at a concentration of from about 1% to about 20% by weight, based upon the total weight of the composition. The oil phase contains at least one water-in-oil emulsifier at a concentration by weight of from about 0.1% to about 20% by weight, based upon the total weight of the composition.

Other features and advantages of the present invention will be apparent from the following more detailed description of the preferred embodiment which illustrates, by way of example, the principles of the invention.

### BRIEF DESCRIPTION OF THE SEVERAL VIEWS OF THE DRAWING

FIG. 1 is a top perspective view showing the visual transparency observed when a neutralized trialkoxysilane is employed, in varying amounts, in a water-in-oil emulsion.

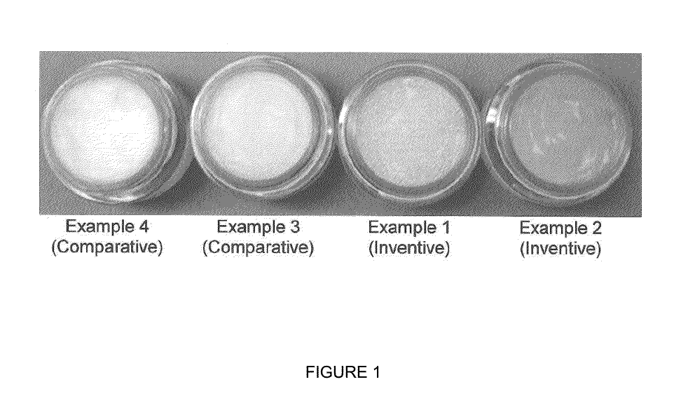

### DETAILED DESCRIPTION OF THE INVENTION

All numbers expressing quantities of ingredients and/or reaction conditions are understood as being modified in all instances by the term “about”, unless otherwise stated.

“Water-in-oil emulsion”, as used herein refers to an emulsion where water phase is dispersed into a continuous oil phase.

“Keratinous substrate,” as used herein, includes but is not limited to skin, hair, and nails.

“Homogenous” means substantially uniform throughout, i.e., a single phase mixture.

“Neutralized trialkoxysilane derivative” as used herein refers to a trialkoxysilane derivative having a pH ranging from about 5 to about 6.

“Translucent”, as used herein, refers to an appearance of the emulsion. Translucency is measured using a chroma meter that measures the absolute numbers in the XYZ color spaces. A test sample is drawn down to give a film of 0.2 mm in thickness and the luminance, Y, is measured on black and white color tiles. The percent (%) translucency is obtained using the following calculation: 100%×(1−(Y_{black}/Y_{white})). A percent translucency of about 85% or greater is considered translucent. A percent translucency of less than about 85% is not considered translucent, and is perceived by human eyes as “opaque”.

In the present application the terms “ambient temperature” or “room temperature” mean a temperature of about 25° C.

In the present application the term “stable” means the emulsion remains intact without phase separation, color and/or odor change over the stability monitoring period and the active ingredients remain solubilized without crystallization or precipitation out of the emulsion.

The composition and method of the present invention can comprise, consist of, or consist essentially of the essential elements and limitations of the invention described herein, as well as any additional or optional ingredients, components, or limitations described herein or otherwise useful in compositions intended for topical application onto keratinous substrates.

It has been surprisingly discovered by the inventors that trialkoxysilane derivatives can be formulated into a stable water-in-oil type emulsion having a translucent appearance, without any negative impact on the sensorial experience to its user. The composition of the present invention can be applied easily and smoothly onto the skin or other keratinous substrate.

### Aqueous Phase

The aqueous phase of the present composition according to the disclosure comprises at least one neutralized trialkoxysilane derivative and water. The aqueous phase of the composition ranges from about 60% to about 92%, or alternatively from about 70% to about 90%, or alternatively from about 80% to about 90% by weight, based upon the total weight of the composition.

### Neutralized Trialkoxysilane Derivatives

Neutralized trialkoxysilane derivatives according to the invention are those corresponding to formula (I), including oligomers thereof:

R₁Si(OR₂)_{z}(R₃)ₓ(OH)_{y} (I)

wherein
R₁ is a linear or branched, saturated or unsaturated C₁-C₂₂, C₃-C₄₀ cycloalkyl or C₆-C₃₀ aromatic hydrocarbon-based chain; optionally substituted with a hydroxyl group, a thiol group, an aryl group (more particularly benzyl), which is substituted or unsubstituted; R₁ possibly being interrupted with a heteroatom (O, S or NH) or a carbonyl group (CO);
R₂ and R₃, which may be identical or different, are a linear or branched alkyl group comprising from 1 to 6 carbon atoms;
y is an integer from 0 to 3;
z is an integer from 0 to 3;
x is an integer from 0 to 2;
wherein z+x+y=3;

“Oligomer,” as used herein, means polymerization products of formula (I) containing from 2 to 10 silicon atoms.

Rather, R2 represents an alkyl group including from 1 to 4 atoms of carbon, preferably a linear alkyl group including from 1 to 4 atoms of carbon, and more preferably the ethyl group.

Preferably, the neutralized trialkoxysilanes may be selected from the group consisting of 3-aminopropyltriethoxysilane (APTES), 3-aminoethyltriethoxysilane (AETES), 3-aminopropylmethyldiethoxysilane, N-(2-aminoethyl)-3-aminopropyltriethoxysilane, 3-(m-aminophenoxy) propyl trimethoxysilane, p-aminophenyltrimethoxysilane, N-(2-aminoethylaminomethyl)-phenethyltrimethoxysilane, oligomers, and mixtures thereof.

More preferably, the neutralized trialkoxysilanes may be selected from the group consisting of 3-aminopropyltriethoxysilane (APTES), 3-aminoethyltriethoxysilane (AETES), their oligomers, and mixtures thereof.

The neutralized trialkoxysilanes are obtained by adding the trialkoxysilanes to water, and then neutralizing them with an acid, such as citric acid for example, to a pH ranging from about 5 to about 6.

Neutralized trialkoxysilanes may be present in the compositions in amounts generally ranging from about 1% to about 20% by weight, preferably from about 2% to about 15% by weight, and most preferably from about 3% to about 10% by weight, based on the total weight of the composition.

### Water

The aqueous phase of the present composition includes water in an amount ranging from about 60% to about 90% by weight, or alternatively about 70% to about 90% by weight or alternatively about 80% to about 90%, based upon the total weight of the composition. The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of thermal water.

### Additional Solvents

In one embodiment, the aqueous phase includes at least one additional solvent. The at least one additional solvent may be employed in an amount ranging from about 0.01% to about 30% by weight, or alternatively about 0.01% to about 20% by weight, or alternatively about 0.01% to about 10% by weight, or alternatively about 0.01% to about 5% by weight, based upon the total weight of the composition. Suitable examples of additional solvents include, but are not limited to, glycerin, propanediol, propylene glycol, butylene glycol, dipropylene glycol, denatured alcohol, and combination thereof.

### Oil Phase

The oil phase present in the composition according to the disclosure includes silicone oil and at least one water-in-oil emulsifier. In one embodiment, the oil phase contains at least one silicone oil in an amount of from about 5% to about 40%, or alternatively about 10% to about 30%, or alternatively about 10% to about 20%, based upon the weight of the composition.

The oil phase of the present disclosure includes silicone oils which are excellent alternatives for traditional oils that are frequently used in personal care formulations. The use of formulations containing traditional oils often leaves the user the impression of greasiness, tackiness, oiliness, or coating. Silicone oils on the other hand, have the benefits of traditional oils, but lack the unfavorable properties and leave a tactile impression of softness, smoothness, and lightness. Non-volatile as well as volatile silicone oils may be used. Non-volatile silicones provide a lasting tactile impression, and tend to form a stable oil layer on the skin. If desired, volatile silicones may be used in combination with non-volatile silicones to impart desired esthetic properties, but preferably the emulsions should contain sufficient non-volatile silicone to provide a skin barrier layer.

Particularly suitable examples of silicone oils, include, but are not limited to dimethicone, dimethiconol, or combinations thereof. In another embodiment, the oil phase may also include cosmetically acceptable additives or adjuvants as well as cosmetic or dermatologic active agents.

### Water-in-Oil Emulsifier

The at least one water-in-oil emulsifier may be selected from self-emulsifying polyether silicone elastomers, polyglyceryl silicone elastomers, polyether dimethicone copolymers, and mixtures thereof. The self-emulsifying silicone elastomers may include functional groups selected from the group consisting of polyglyceryl, polyethylene glycol, or polypropylene glycol. Suitable examples of commercially available self-emulsifying silicone elastomer water-in-oil emulsifiers, include, but are not limited to, dimethicone (and) dimethicone/PEG-10/15 crosspolymer, sold under the tradename KSG-210, and dimethicone (and) dimethicone/polyglycerin-3 crosspolymer, sold under the tradename of KSG-710, both available from Shin-Etsu. Examples of water-in-oil/silicone emulsifiers may also include, but are not limited to, PEG-10 dimethicone sold under the tradename KF-6017 from Shin-Etsu, dimethicone (and) PEG/PPG-18/18 dimethicone sold under the tradename ES-5226 DM from Dow Corning Corporation, PEG-9 polydimethylsiloxyethyl dimethicone (and) PEG-9 available under the tradename KF-6028, and lauryl PEG-9 polydimethylsiloxyethyl dimethicone available under the tradename KF-6038, both available from Shin-Etsu.

The water-in-oil emulsifier is advantageously present at a concentration of about 0.1% to about 20%, or alternatively about 0.5% to about 10%, or alternatively about 1% to about 5% by weight, based upon the weight of the composition.

### Co-Emulsifier

In one embodiment, the oil phase further includes co-emulsifiers, such as, polyol alkyl esters of glycerol and sorbitan esters, including for example polyglyceryl-4 isostearate, such as the product sold under the tradename Isolan® GI 34 by Evonik, polyglyceryl-3 diisostearate, sold under the tradename Lameform® TGI by BASF, sorbitan isostearate, such as the product sold under the tradename Arlacel™ 987 by Croda, sorbitan glyceryl isostearate, such as the product sold under the tradename Arlacel™ 986 by Croda, and mixtures thereof. The co-emulsifiers facilitate further stability of the composition. The co-emulsifier is advantageously present in an amount from about 0.1% to about 6% by weight, more particularly from about 0.5% to about 5% by weight, based on the weight of the composition.

### Optional Powders

The composition of the present disclosure may optionally include powders. The optional powders provide formulas that are smoother and softer on the skin. Representative powders include, but are not limited to, talc, mica, magnesium carbonate, calcium carbonate, magnesium silicate, aluminum magnesium silicate, silica, titanium dioxide, zinc oxide, red iron oxide, yellow iron oxide, black iron oxide, polyethylene powder, methacrylate powder, polystyrene powder, silk powder, crystalline cellulose, starch, titanated mica, iron oxide titanated mica, bismuth oxychloride, and the like. Additional powders include, but are not limited to, inorganic powders such as gums, chalk, Fuller's earth, kaolin, sericite, muscovite, phlogopite, synthetic mica, lepidolite, biotite, lithia mica, vermiculite, aluminum silicate, starch, smectite clays, alkyl and/or trialkyl aryl ammonium smectites, chemically modified magnesium aluminum silicate, organically modified montmorillonite clay, hydrated aluminum silicate, fumed aluminum starch octenyl succinate barium silicate, calcium silicate, magnesium silicate, strontium silicate, metal tungstate, magnesium, silica alumina, zeolite, barium sulfate, calcined calcium sulfate (calcined gypsum), calcium phosphate, fluorine apatite, hydroxyapatite, ceramic powder, metallic soap (zinc stearate, magnesium stearate, zinc myristate, calcium palmitate, and aluminum stearate), colloidal silicone dioxide, and boron nitride; organic powder such as polyamide resin powder (nylon powder), cyclodextrin, methyl polymethacrylate powder, copolymer powder of styrene and acrylic acid, benzoguanamine resin powder, poly(ethylene tetrafluoride) powder, and carboxyvinyl polymer, cellulose powder such as hydroxyethyl cellulose and sodium carboxymethyl cellulose, ethylene glycol monostearate; inorganic white pigments such as magnesium oxide. A representative powder includes, for example, polymethylsilsesquioxane or silica silyate. Powders may be present in the compositions in amounts generally ranging from about 0.1% to about 10% by weight, based on the total weight of the composition.

The composition may further contain at least one auxiliary ingredient chosen from humectants, preservatives, buffering agents such as salts, and the like.

### Translucency

The water-in-oil emulsions of the present disclosure are translucent in appearance. For each of the examples, the translucency is measured using a Konica Minlolta CR-400 chroma meter. The chroma meter measures the absolute numbers in the XYZ color spaces. To measure the translucency, a 0.2 mm film of a sample is drawn down on a 50 μm thick polyethylene substrate, for example, and the luminance, Y, is measured on black and white contrast film. The percent (%) transparency is obtained using the measured Y_{black} and Y_{white} value and the following formula: 100%×(1−Y_{black}/Y_{white}). A percent translucency of about 85% or greater is considered translucent. A percent translucency of less than about 85% is not considered translucent. Inventive Examples all have a percent translucency of about 85% or greater and are deemed to be translucent. Conversely, the Comparative Examples have a percent translucency of less than 85% and are thus considered opaque.

According to one embodiment of the present invention, there is provided a composition containing: (a) an aqueous phase having: (i) from about 60 to 90% by weight of water; (ii) from about 1% to 20% by weight of a neutralized trialkoxysilane, preferably aminopropyl triethoxysilane; (iii) an acid in the amount sufficient to maintain the pH of the aqueous phase in the range of about 5 to about 6; (iv) optionally, from about 0.01% to about 30% by weight of a co-solvent, preferably glycerin, and (b) an oil phase having: (v) at least one silicone oil, preferably dimethicone, in an amount of from about 5% to about 40% by weight; (vi) at least one emulsifier chosen from self-emulsifying polyether silicone elastomers, polyglyceryl silicone elastomers, polyether dimethicone copolymers, and mixtures thereof, in an amount of from about 0.1% to about 20% by weight; and (vii) optionally, one co-emulsifier, all weights based on the total active weight of the composition, wherein the composition is translucent in appearance.

According to another embodiment of the present invention, there is provided a method of making a composition involving: (a) providing an aqueous phase having: (i) from about 60 to 90% by weight of water; (ii) from about 1% to 20% by weight of a neutralized trialkoxysilane, preferably aminopropyl triethoxysilane; (iii) an acid in the amount sufficient to maintain the pH of the aqueous phase in the range of about 5 to 6; (iv) optionally, from about 0.01% to 30% by weight of a co-solvent, preferably glycerin, (b) providing an oil phase having: (v) at least one silicone oil, preferably dimethicone, in an amount of from about 5% to 40% by weight; (vi) at least one emulsifier chosen from self-emulsifying polyether silicone elastomers, polyglyceryl silicone elastomers, polyether dimethicone copolymers, and mixtures thereof, in an amount of from about 0.1% to 20% by weight; and (vii) optionally, one co-emulsifier, all weights based on the total active weight of the composition, and (c) mixing (a) and (b) to form the composition, wherein the composition is translucent in appearance.

The present invention will be better understood in view of the examples, all of which are illustrative only and are not intended to limit the scope of the invention in any way.

### EXAMPLES

In making each of examples in Tables 1 and 2, the following procedure is used. The ingredients of Phase A (oil phase) were placed in a main beaker and mixed with a propeller mixer at about 300 RPM until the ingredients were well dispersed. The ingredients of Phase B1 (aqueous phase of APTES, if present) were placed in a side beaker and mixed with a propeller mixer until fully dissolved, giving a clear solution. Phase B2 was added to the side beaker and mixed until fully dissolved. Contents of the side beaker were adjusted to a pH of about 5 to 5.5. The combined aqueous phase mixture (Phase B) was slowly added to the oil phase (Phase A) using a propeller mixer. As the viscosity of the mixture increased, the stirring speed was increased to about 1000 rpm, until a homogeneous emulsion was obtained.

All examples (inventive and comparative) were monitored for emulsion stability for a period of 8 weeks at 5° C., 25° C., 37° C. and 45° C. and after 10 cycles of freeze/thaw (cycled from −20° C. to 25° C. for 12 hours at each temperature). At each end point, the aspect of the test formulas was examined for signs of instability such as graininess, inhomogeneous and/or phase separation by visual assessment and/or microscopic imaging. Viscosity was taken at 4 and 8 weeks at 25° C. and compared to the initial values. Viscosity of the emulsion was measured using a Brookfield Viscometer, using Heliopath spindle T-D and speed set at 10 rpm. The spindle was allowed to oscillate in the test sample, and the measurements were taken after one minute.

All examples outline herein passed the stability protocol as described above. However the visual aspect of each test formula varied depending on the concentration of trialkoxysilane derivatives.

TABLE 1
Examples

| | | Inventive | | Comparative | |
|---|---|---|---|---|---|
| Phase | INCI US Name | Ex. 1 | Ex. 2 | Ex. 3 | Ex. 4 |
| A | DIMETHICONE (and) | 5 | 5 | 5 | 5 |
| | DIMETHICONE/PEG-10/15 | | | | |
| | CROSSPOLYMER | | | | |
| A | PEG-10 DIMETHICONE | 0.1 | 0.1 | 0.1 | 0.1 |
| A | DIMETHICONE (and) DIMETHICONOL | 1 | 1 | 1 | 1 |
| A | DIMETHICONE | 10 | 10 | 10 | 10 |
| B1 | WATER | 51.45 | 44.45 | 58.45 | 73.45 |
| B1 | 3-AMINOPROPYL TRIETHOXYSILANE | 5 | 10 | — | — |
| | (APTES) | | | | |
| B1 | CITRIC ACID | 2 | 4 | — | — |
| B2 | GLYCERIN | 15 | 15 | 15 | 0 |
| B2 | PHENOXYETHANOL | 0.5 | 0.5 | 0.5 | 0.5 |
| B2 | DISODIUM EDTA | 0.1 | 0.1 | 0.1 | 0.1 |
| B2 | SODIUM CITRATE | 0.2 | 0.2 | 0.2 | 0.2 |
| B2 | SODIUM CHLORIDE | 0.8 | 0.8 | 0.8 | 0.8 |
| C | ALCOHOL DENAT. | 3 | 3 | 3 | 3 |
| C | PROPANEDIOL | 5 | 5 | 5 | 5 |
| C | p-ANISIC ACID | 0.15 | 0.15 | 0.15 | 0.15 |
| C | FRAGRANCE | 0.2 | 0.2 | 0.2 | 0.2 |
| D | SILICA SILYLATE | 0.5 | 0.5 | 0.5 | 0.5 |
| | Total (%): | 100 | 100 | 100 | 100 |
| | Texture: | Translucent | | Opaque white | |
| | | cream-gel | | cream | |
| | Initial Viscosity (cP): | 47,000 | 40,000 | 53,000 | 60,000 |
| | Transparency (%): | 85.0 | 87.5 | 82.2 | 74.7 |

TABLE 2
Examples

| | | Inventive | Comparative | |
|---|---|---|---|---|
| Phase | INCI Name | Ex. 5 | Ex. 6 | Ex. 7 |
| A | DIMETHICONE | 13.5 | 18.9 | 18.9 |
| A | DIMETHICONE (and) DIMETHICONE | 10 | 10 | 10 |
| | CROSSPOLYMER | | | |
| A | POLYGLYCERYL-4 ISOSTEARATE | 4 | 4 | 4 |
| A | DIMETHICONE (and) PEG/PPG-18/18 | 2 | 2 | 2 |
| | DIMETHICONE | | | |
| A | PEG-9 POLYDIMETHYLSILOXYETHYL | 1.5 | 1.5 | 1.5 |
| | DIMETHICONE (and) PEG-9 | | | |
| A | POLYSORBATE 20 | 0.5 | 0.5 | 0.5 |
| B1 | WATER | QS to 100 | QS to 100 | QS to 100 |
| B1 | 3-AMINOPROPYL TRIETHOXYSILANE | 10 | — | — |
| | (APTES) | | | |
| B1 | CITRIC ACID | 3.1 | — | — |
| B2 | GLYCERIN | 10 | 10 | — |
| B2 | MAGNESIUM SULFATE | 1 | 1 | 1 |
| B2 | PHENOXYETHANOL | 0.5 | 0.5 | 0.5 |
| B2 | DISODIUM EDTA | 0.1 | 0.1 | 0.1 |
| B2 | CITRIC ACID | QS pH of Phase B to 5 | | |
| B2 | SODIUM HYDROXIDE | | | |
| | Total (%): | 100 | 100 | 100 |
| | Texture: | Translucent | Opaque white | |
| | | cream-gel | cream | |
| | Initial Viscosity (cP): | 25,000 | 18,000 | 14,000 |
| | Transparency (%): | 87.0 | 80.1 | 75.0 |

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A composition comprising:
(a) an aqueous phase comprising:
from about 60% to about 90% by weight of water;
(ii) from about 1% to about 20% by weight of a neutralized trialkoxysilane derivative;
(iii) an acid in an amount sufficient to maintain the pH of the aqueous phase in the range of about 5 to about 6;
(iv) optionally, a co-solvent; and
(b) an oil phase comprising:
(i) from about 5% to about 40% by weight at least one silicone oil;
(ii) from about 0.1% to 20% by weight of at least one emulsifier chosen from self-emulsifying polyether silicone elastomers, polyglyceryl silicone elastomers, polyether dimethicone copolymers, and mixtures thereof; and
(iii) optionally, one co-emulsifier;
said composition being translucent; all weights being based on the total weight of the composition.

2. A composition comprising:
(a) an aqueous phase comprising:
(i) from about 60% to about 90% by weight of water;
(ii) from about 1% to about 20% by weight of a neutralized trialkoxysilane derivative selected from the group consisting of 3-aminopropyltriethoxysilane (APTES), 3-aminoethyltriethoxysilane (AETES), 3-aminopropylmethyltriethoxysilane, N-(2-aminoethyl)-3-aminopropyltriethoxysilane, 3-(m-aminophenoxy) propyl trimethoxysilane, p-aminophenyltrimethoxysilane, N-(2-aminoethylaminomethyl)-phenethyltrimethoxysilane, oligomers, and mixtures thereof;
(iii) an acid in an amount sufficient to maintain the pH of the aqueous phase in the range of about 5 to about 6;
(iv) optionally, a co-solvent; and
(b) an oil phase comprising:
(i) from about 5% to about 40% of at least one silicone oil;
(ii) from about 0.1% to 20% of at least one emulsifier chosen from self-emulsifying polyether silicone elastomers, polyglyceryl silicone elastomers, polyether dimethicone copolymers, and mixtures thereof; and
(iii) optionally, a co-emulsifier;
said composition having a translucency equal to or greater than 85%; all weights being based on the total weight of the composition.

3. The composition of claim 2 comprising a co-solvent.

4. The composition of claim 3 wherein the co-solvent is present in an amount from about 0.01% to about 30%.

5. The composition of claim 4 wherein the co-solvent is selected from the group consisting of glycerin, propanediol, propylene glycol, butylene glycol, dipropylene glycol, denatured alcohol, and mixtures thereof.

6. The composition of claim 5 wherein the silicone oil is selected from the group consisting of dimethicone, dimethiconol, and mixtures thereof.

7. The composition of claim 6 wherein the emulsifier is selected from the group consisting of dimethicone (and) dimethicone/PEG-10/15 crosspolymer, dimethicone (and) dimethicone/polyglycerin-3 crosspolymer, PEG-10 dimethicone, dimethicone (and) PEG/PPG-18/18 dimethicone, PEG-9 polydimethylsiloxyethyl dimethicone (and) PEG-9, lauryl PEG-9 polydimethylsiloxyethyl dimethicone, and mixtures thereof.

8. The composition of claim 7 comprising from about 0.1% to about 6% of a co-emulsifier.

9. The composition of claim 8 wherein the co-emulsifier is selected from the group consisting of polyglyceryl-4 isostearate, polyglyceryl-3 diisostearate, sorbitan isostearate, sorbitan glyceryl isostearate, and mixtures thereof.

10. The composition of claim 9 further comprising from about 0.1% to about 10% by weight of powders.

11. The composition of claim 7 having a translucently equal to or greater than 85%.

12. The composition of claim 11 that is a cosmetic composition.

13. A method of making an translucent emulsion comprising:
(a) providing an aqueous phase having:
(i) from about 60% to 90% by weight of water;
(ii) from about 1% to 20% by weight of a neutralized trialkoxysilane derivative;
(iii) an acid in the amount sufficient to maintain the pH of the aqueous phase in the range of about 5 to 5.5; and
(iv) optionally, from about 0.01% to 30% by weight of a co-solvent;
(b) providing an oil phase having:
(i) at least one silicone oil in an amount of from about 5% to 40% by weight;
(ii) at least one emulsifier chosen from self-emulsifying polyether silicone elastomers, polyglyceryl silicone elastomers, polyether dimethicone copolymers, and mixtures thereof, in an amount of from about 0.1% to 20% by weight; and
(iii) optionally, one co-emulsifier;
all weights based on the total active weight of the composition; and
(c) mixing the aqueous phase (a) and oil phase (b) to form the emulsion.

14. The composition of claim 1, wherein the neutralized trialkoxysilane derivative has the Formula (I):
R₁Si(OR₂)_{z}(R₃)ₓ(OH)_{y} (I)
wherein
R₁ is a linear or branched, saturated or unsaturated C₁-C₂₂, C₃-C₄₀ cycloalkyl or C₆-C₃₀ aromatic hydrocarbon-based chain; optionally substituted with a hydroxyl group, a thiol group, an aryl group (more particularly benzyl), which is substituted or unsubstituted; R₁ possibly being interrupted with a heteroatom (O, S or NH) or a carbonyl group (CO);
R₂ and R₃, which may be identical or different, each is a linear or branched alkyl group comprising from 1 to 6 carbon atoms;
y is an integer from 0 to 3;
z is an integer from 0 to 3;
x is an integer from 0 to 2;
wherein z+x+y=3.

15. The composition of claim 14, wherein the neutralized trialkoxysilane derivative is an oligomer comprising a polymerization product of Formula (I), comprising from 2 to 10 silicon atoms.

16. The composition of claim 15, wherein R2 represents an alkyl group including from 1 to 4 atoms of carbon.

17. The composition of claim 16, wherein R2 is a linear alkyl group including from 1 to 4 atoms of carbon.

18. The composition of claim 17, wherein R2, comprises an ethyl group.

19. The composition of claim 14, wherein the neutralized trialkoxysilane is selected one or more of 3-aminopropyltriethoxysilane (APTES), 3-aminoethyltriethoxysilane (AETES), 3-aminopropylmethyltriethoxysilane, N-(2-aminoethyl)-3-aminopropyltriethoxysilane, 3-(m-aminophenoxy) propyl trimethoxysilane, p-aminophenyltrimethoxysilane, N-(2-aminoethylaminomethyl)-phenethyltrimethoxysilane, and oligomers thereof.

20. The composition of claim 14, wherein the neutralized trialkoxysilane derivative is obtained by adding a trialkoxysilane to water, and neutralizing with an acid to a pH ranging from about 5 to about 6.
